(19) United States
(12) Patent Application Publication
(10) Pub. No.: US 2009/0037031 A1
(43) Pub. Date: Feb. 5, 2009

(54) System Comprising a Plurality of Incubators

(75) Inventors: Christian GEORGE, Wolfratshausen (DE); Michel MOMBOISSE, Munchen (DE); Torsten BURDACK, Munchen (DE); Ralf PUKLOWSKY, Munchen (DE); Giuseppe MARINO, Munchen (DE); Guenter TENZLER, Munchen (DE)

Correspondence Address: MYERS WOLIN, LLC, 100 HEADQUARTERS PLAZA, North Tower, 6th Floor, MORRISTOWN, NJ, 07960-6834, US

(73) Assignee: INHECO INDUSTRIAL HEATING AND COOLING GMBH, Martinsried (DE)

(21) Appl. No.: 12/025,094

(22) Filed: Feb. 4, 2008

Related U.S. Application Data
(63) Continuation of application No. PCT/EP2006/007533, filed on Jul. 29, 2006.

(30) Foreign Application Priority Data
Aug. 4, 2005 (DE) 10 2005 036 763.1

(51) Int. Cl. G05D 23/00; C12M 3/00

(52) U.S. Cl. 700/300; 435/303.1

(57) ABSTRACT

A system comprised of a number of incubators each having a housing, a sample chamber placed inside the housing, a temperature control unit, and control electronics that control the temperature control unit. The sample chambers of the incubators can be heated and/or cooled. The housings of the incubators each have a closeable access opening for loading and unloading the sample chamber. The housings permit the incubators to be vertically stacked. The incubators each have a bus system that interacts with its control electronics. The bus systems of the incubators are interconnected via corresponding connecting elements, and the system comprises a central control unit that is connected to the bus system of one of the incubators of the system (master incubator). Over the bus system architecture, an individual controlling of the control electronics of all incubators of the system is made possible by a central control unit.

This is a continuation of PCT/EP2006/007533, filed Jul. 29, 2006, which claims priority to German Patent Application 10 2005 036 763.1 filed Aug. 4, 2005, the contents of each of which are incorporated herein by reference.

### FIELD OF THE INVENTION

The present invention relates to a system comprising a plurality of incubators, each encompassing a housing, a sample chamber disposed inside the housing for receiving a sample container, a temperature-regulating unit and an electronic controller that controls the temperature-regulating unit, wherein the sample chambers of the incubators can be heated and/or cooled by means of the temperature-regulating units and the housings of the incubators each have a closable access opening for loading and unloading the sample chamber, and the housings of the incubators permit vertical stacking of the incubators.

Such incubators are preferably used for samples in the disciplines of biology, biotechnology, pharmacy, diagnostics or chemistry that are to be exposed in the sample chamber of the incubator to well-defined environmental conditions for a certain time period. For this purpose, the samples in a sample container are introduced into the sample chamber of the incubator. In the case of liquid samples, the sample containers are liquid containers having one or more subcontainers, for example of the type known as “microplates” or “deep-well microplates”.

### BACKGROUND

A system comprising a plurality of incubators for receiving “microplates” having the generic features of the present invention is known, for example, from U.S. Pat. No. 6,518,059 B1. Therein a plurality of incubators having identical functionality can be stacked one above the other. According to two alternative embodiments, it is further provided that the temperature in the sample chambers of the incubators can be regulated separately in each incubator by means of its own temperature-control unit or can be controlled by means of the temperature-control unit of what is known as a master incubator. In the latter case, the incubators can be operated only at substantially the same temperature in the sample chamber.

Starting from the already known prior art, it is the object of the present invention to provide a modular system that can be used as universally as possible, that comprises a plurality of incubators of the class in question, that can be expanded or reassembled as simply as possible and that achieves—even in combination with further automation instruments—the highest possible functionality, which offers the possibility of exposing different samples simultaneously to different environmental conditions and ultimately makes it possible to combine incubators of different designs flexibly as one system.

### SUMMARY

This object is achieved by a system of the class in question having the further features of claim 1, specifically by the fact that the incubators respectively have a bus system cooperating with their electronic controllers, wherein the bus systems of the incubators are connected to one another via appropriate connecting elements, the system encompasses a central control unit, which is connected to the bus system of one of the incubators of the system (master incubator), and the electronic controllers of all incubators of the system can be activated individually by the central control unit by virtue of the architecture of the bus system.

The inventive system is therefore characterized in that only one incubator—known as the master incubator—has to be connected to a central control unit. Bus systems installed in the incubators and connected to one another permit the electronic controllers of all incubators of the system to be activated separately and individually by the central control unit. Thus it is possible—while at the same time achieving modularity—to expose samples in different sample chambers simultaneously to different temperatures. The inventive system can be expanded extremely simply, since an incubator to be newly added to the system does not have to be connected directly to the central control unit in order to ensure that it can be individually activated. Instead, only the bus system cooperating with its electronic controller has to be connected to the bus system of an incubator already present beforehand in the system in order to permit the electronic controller of the newly added incubator to be activated by the central control unit by virtue of the architecture of the bus system.

Furthermore, the inventive system affords the user the possibility, not available in the prior art, of also integrating incubators of different designs and functionalities into the system, provided they have the features defined in more detail in claim 1, especially a bus system that cooperates with their electronic controller and is mutually compatible.

For example, incubators whose sample chambers can only be heated by means of a temperature-regulating unit can be augmented simply in this way by an incubator whose sample chamber can only be cooled.

Obviously the incubators may also differ as regards other additional functionalities, as will be examined in more detail hereinafter.

In the specific configuration of the bus systems of the incubator and linking of the bus systems to the respective electronic controllers, the person skilled in the art will make use of the prior art that is definitive for bus systems, from which art there are known bus systems having the most diverse technical embodiments.

Advantageously, such incubators added newly to the system are automatically recognized by the central control unit, which if necessary immediately assigns them a bus-system address by means of which they can be activated. Furthermore, the bus system of each incubator advantageously has means for identifying its type or its functionality. By virtue of the foregoing features, the system achieves what is known as “plug-and-play” functionality, which permits particularly simple initialization of an added incubator. Furthermore, it can be provided, if the spatial situation of a newly added incubator necessarily permits on the basis of its connection to the bus system (see hereinafter), that the incubator will immediately be assigned appropriate spatial coordinates, thus greatly facilitating cooperation with an automated robot system, for example for the purpose of loading and unloading the sample chambers of the incubators.

According to a first advantageous configuration of the inventive system, it can be provided that two neighboring incubators of the system can be connected by means of two respective connecting elements in the form of mating plug-and-socket elements, wherein the plug-and-socket elements of at least some of the incubators are disposed opposite one another on the upper side and underside of their housing, in such a way that they form a direct plug-and-socket connection with likewise mating plug-and-socket elements of the incubator disposed below them and/or stacked on top of them.

For this purpose it is obvious that all incubators that are present in the stack and that respectively have an upper and lower “neighbor incubator” will preferably be equipped with two such connecting elements. The two incubators bounding the stack at the top and bottom actually need an appropriate connecting element only on their upper side or underside. For expansion capability of the system, however, it has proved advantageous for the uppermost incubator of the stack also to have two corresponding connecting elements in the form of mating plug-and-socket elements, since then at least one further incubator—having at least one corresponding plug-and-socket element on its underside—can also be added later by simply stacking it on top.

In the configuration of the system described in the foregoing, there is no need to provide and connect an additional connecting cable. Instead, connection of the bus systems is already achieved directly when a newly added incubator is stacked onto the already existing stack. This also makes it possible in particular for an added incubator to be automatically assigned the spatial coordinates corresponding to its spatial situation, since it is necessarily disposed directly above the uppermost incubator of a stack and its position relative to the master incubator can be deduced from the already known geometric dimensions of the incubator—possibly depending on its design, which can be identified via the bus system.

In view of the configuration and arrangement of the connecting elements of the incubators as described in the foregoing, it is obviously especially advantageous—in the sense of reassembly of the system as simply as possible—if the master incubator connected to the central control unit represents the lowermost incubator in the system comprising a plurality of incubators stacked according to the invention. For this purpose its bus system is advantageously connected to the central control unit, via a plug-and-socket element on the underside of its housing if it is so equipped, or if necessary even via another connecting element.

In a further advantageous configuration of the system, at least some of the incubators have a drawer for loading and unloading the sample chamber, which drawer is guided in linear guides and by means of a motor can be withdrawn through the access opening—which is preferably disposed on the front side—from the housing of the incubator and reinserted in the said opening.

Thereby the incubators of the system can be loaded and unloaded in automated manner. Advantageously, the incubator therefore also has an electronic controller—which preferably can be controlled via the bus system—for the drive unit of the drawer, which controller can again preferably be the electronic controller that is already present for control of the temperature-regulating unit and that thus assumes a further function. The samples or sample containers can then be supplied to and deposited in the completely withdrawn drawer by means of a robot system—which is preferably controlled by the central control unit. For the topmost incubator of a stack, it may be permissible to dispense with such a drawer, since such an incubator could also be loaded and unloaded in automated manner through an access opening disposed at its upper side.

The drawer of the incubators must be suitable for supporting the sample or the sample container, and at the same time it can form the bottom of the sample chamber. For this purpose it can be configured, for example, as a continuous surface. However, this is not mandatory; the drawer may also be, for example, a frame-like holder, which supports the sample containers only in its rim region.

In particular, it proves advantageous if the drawer is mounted so as to be vertically fixed in both linear guides, whereas in the horizontal direction transverse to its direction of movement it is fixed in one of the linear guides and mounted loosely in the other linear guide. This permits some thermal expansion of the drawer and thus prevents jamming when expansion occurs.

In yet another preferred configuration of the system, the drawer in its completely withdrawn end position bears against at least one or more stops of the incubator in question. In the case of stops having appropriately precise tolerances, this is particularly advantageous in conjunction with an automated robot system for handling the sample containers, since the position of the completely withdrawn drawer is still very accurately reproducible by virtue of the at least one stop even after many opening and closing cycles, thus ensuring that the sample containers will not be incorrectly grasped or incorrectly deposited by the robot.

According to an advantageous improvement of the system, the front access opening to the sample chamber of the incubator is closed by a shutter fastened on the end face of the drawer. Thus, in contrast to a shutter attached to the housing of the incubator, it does not have to be provided with a separate opening mechanism. Also, the closing movement of the drawer can be converted into active closing of the shutter.

Another preferred improvement of the system provides that the shutter occupies a substantially vertical position that closes the access opening when the drawer is inserted and is fastened to the drawer by means of a horizontal spindle, so as to pivot out of the way into substantially horizontal position when the drawer is opened. For this purpose, an initial tension can be applied to the shutter, for example by a spring means. Thus, when it is in its substantially horizontal position, it does not interfere with a gripper arm that may be used by a robot system for handling the sample containers.

On the shutter side, the pivot spindle between drawer and shutter is advantageously disposed at a distance from the lower and upper rim—as viewed in vertical position—of the shutter. By this arrangement, which is disposed at a distance from the rim and is therefore more central, a closing force distributed substantially evenly over the shutter can be applied when the access opening of the housing is closed by inserting the drawer. Furthermore, the space needed for the shutter to be pivoted out of the way is smaller than in the case of a rim-mounted pivot spindle.

For this purpose the shutter must be restored to its upright or vertical position by suitable means when the drawer is closed, in order to close the access opening to the sample chamber properly. This can be advantageously achieved by an appropriate curved guide on the housing, against which guide part of the shutter is stopped during closing of the drawer and is deflected in such a way that the shutter becomes upright once again.

By the configuration of the system explained in the foregoing, this system can cooperate with robot or gripper systems of different designs. Especially for microplates or other standardized, elongated sample containers, there are already known various robot systems that grip the sample containers along their long or short sides in what is known as portrait or landscape mode in order to transport them. The linearly guided drawer with shutter attached on the front side permits the labware to be gripped and deposited in portrait mode. When the shutter is pivoted out of the way into substantially horizontal position, sample containers can additionally be gripped in landscape mode.

Advantageously, the incubators of the inventive system have means for applying a certain closing force to the closed shutter. For this purpose, it is possible, for example, to supply a drawer drive motor designed as an electric motor with a constant motor current during the closed condition, to the effect that the shutter is maintained closed by the motor force. Alternatively, it is also preferably possible to provide magnetic elements—which if necessary can be switched—on the incubator and/or on the shutter, in order to ensure sufficiently sealed closing of the shutter.

In a further advantageous configuration of the system, the temperature-regulating unit of at least one incubator encompasses a heating film disposed on the bottom of the sample chamber, with which film the sample chamber and the sample present therein can be heated.

In an advantageous improvement, the temperature-regulating unit encompasses either a two-zone heating film or at least two heating films, with which at least two zones can be heated separately. In this case, one of the zones advantageously covers a peripheral rim region of the bottom of the sample chamber, while the second zone covers the inner central region of the bottom of the sample chamber. During heating of the sample chamber to the desired operating temperature, the entire heating power of all heating zones is advantageously available, whereas only a heating zone located in the outer rim region of the sample chamber remains in operation regulated to the setpoint temperature in order to maintain an already achieved operating temperature. In this way a particularly homogeneous temperature distribution inside the sample chamber—and thus also inside the sample itself—can be achieved, since the greatest heat losses occur in the rim regions of the sample chamber. Furthermore, the heating power density in the rim region preferably can be additionally adapted so as to compensate for different heat transmission conditions at the corners of the sample chamber or along the long and short sides.

The temperature-regulating unit of at least one incubator of the inventive system can advantageously also encompass a cooling group or a combined cooling/heating group. Advantageously only the bottom and/or side walls of the sample chamber can be directly cooled in this way, whereas the cover of the sample chamber is not directly cooled. Furthermore, Peltier modules based on the thermoelectric effect can be advantageously used for this purpose, since they permit either cooling or heating depending on the sign of the operating voltage. A largely uniform temperature distribution can be achieved by cooling the side walls—by analogy with heating an outer rim region of the bottom of the sample chamber. In contrast to the case of a heating film, it is very difficult with Peltier modules to cool the rim regions of the bottom of the sample chamber more intensively. By the fact that the cover of the sample chamber is expressly not cooled directly, condensate formation on the cover and related dripping of condensate onto the sample(s) present in the sample container is effectively prevented.

In the interest of the smallest possible overall height of the incubators of the system, it can be further provided that the bottom and/or side walls of the sample chamber is/are spatially separated from the actual cooling group or the combined heating/cooling group, but are in communication therewith by what are known as heat pipes. The temperature-regulating unit then advantageously encompasses a cooling or heating group fan unit as well as a cooling or heating flange, which on one side is in communication with the cooling or heating group and on the other side is in contact with the heat pipes. In this way the cooling or heating group can also be positioned at the side or behind the sample chamber in the incubator. Advantageously, the cooling or heating flange is disposed at the same height as a bottom to be cooled or heated. Such substantially horizontal operation of the heat pipe(s) results in particularly good heating or cooling capacity.

In a further advantageous configuration of the system, the insulation of at least one of the heatable and/or coolable incubators encompasses a sandwich structure comprising at least two different insulating systems, the outer of which is designed as vacuum insulation. The inner insulating system can advantageously be a layer of solid plastic or a layer of closed-cell foam rubber. Also preferably, especially in regard to the smallest possible thickness of the insulation and thus a particularly small overall height of the incubators, there are provided in total three insulating layers comprising—from inside to outside—solid plastic, closed-cell foam rubber and vacuum insulation. A small overall height of the incubators is also extremely positive for the system as such, since in this way a smaller stack height is achieved for a given number of incubators, thus in turn increasing the stability of such a stack. Good insulation in the present system is also particularly important because the neighboring incubators in a stack may be required to operate at different temperatures. Thus mutual influences of neighboring incubators can be reduced by the insulation described in the foregoing.

Closed-cell foam rubber achieves its good insulating effect by air cells included in the rubber. Vacuum insulation preferably encompasses an insulating core of microporous silica evacuated to a pressure of below 5 mbar and then sealed vacuum-tight with an envelope of metal-vapor-deposited multi-layer film; in a thickness of only 4 mm, this has proved adequate in practice—without further insulating materials—up to temperatures of approximately 60° C. in the sample chamber. In the inventive system, however, the system can advantageously also include at least one incubator whose temperature-regulating unit reaches a temperature of up to 80° C. or even higher (such as 100° C. or 105° C.) in the sample chamber. In the case of the two-layer or three-layer insulation described in the foregoing, the inner layers of plastic and/or rubber are advantageously dimensioned such that in normal operation they reduce an internal temperature of 80° C. or higher in the sample chamber to 60° C. at the outer surface of the second insulating layer, thus allowing the vacuum insulation to insulate the residual heat adequately.

In yet another advantageous configuration of the inventive system, in which the incubators have a drawer for loading and unloading the sample chamber, the drawer encompasses a holder for a sample container, the holder being guided by a slide guide. This slide guide brings about vertical lowering of the holder during the last portion of the horizontal insertion travel of the drawer into the housing, the sample container being deposited on an intermediate plate or on the bottom of the sample chamber. Deposition of the sample containers on the bottom of the sample chamber proves particularly appropriate if, for example, the bottom is in static communication with a temperature-regulating unit by means of heat pipes and thus cannot be used itself as part of the drawer for loading and unloading the sample chamber. In this way there is achieved the advantage that the sample chamber is deposited directly on the coolable and/or heatable bottom of the sample chamber, thus permitting better heat transfer to the sample.

However, the sample or the sample container can also be deposited on an intermediate plate, which is different from the bottom of the sample chamber. Such a plate—especially in the case that the underside of the sample containers being used is not flat—can then be configured as an adapter plate to ensure the best possible transfer of heating and/or cooling capacity of a heatable and/or coolable bottom plate, by the fact that its geometry is matched on its underside to the bottom of the sample chamber and on its upper side to the geometry of the sample containers to be introduced into the sample container. In such cases the intermediate plate is advantageously made of aluminum or of a material that has comparable or better thermal conductivity.

Alternatively, however, it can also be provided that the intermediate plate is the upper side of a shaker table, which is integrated into the incubator and with which the sample can be shaken. For this purpose the intermediate plate is advantageously disposed such that it can be moved in the horizontal plane and be excited to vibrations in the horizontal plane by means of a suitable drive system. This shaker unit is preferably—as is otherwise advantageously the case for all electronic components of the incubators of the inventive system—controlled by the electronic controller of the incubator, and thus can also be activated by the central control unit via the bus system.

Besides the means described heretofore for influencing environmental conditions for the samples located in the sample chamber (such as temperature-regulating unit for the temperature, optional shaker table for initiating a horizontal shaking movement), at least one incubator of the inventive system can also have at least one further means for influencing further environmental conditions. Such means include in particular an appropriate light source for illuminating the samples with UV or infrared light, an antenna for irradiating samples with electromagnetic fields in various frequency ranges, a gas inlet and gas outlet aperture, which can be electronically switched if necessary, for admitting nitrogen (N2), for example, or another gas to the samples, means for raising or lowering the relative humidity in the sample chamber, etc. In the case of the present means for influencing the environmental conditions in the sample chamber, it can be provided in particular in an advantageous configuration that these can also be activated by the electronic controller and therefore by the central control unit. Especially in conjunction with the inventively provided bus system, which advantageously recognizes the design and functionality of various incubators to be connected to the system automatically via “plug-and-play” capability, the inventive system proves in this way to be extremely user-friendly.

Obviously the various advantageous configurations of the system or of the incubators described in detail in the foregoing can be combined in any desired way.

In addition to the inventive system comprising a plurality of incubators, stand-alone protection for appropriately configured incubators is also claimed.

In particular, this relates first to an incubator (master incubator) encompassing a housing, a sample chamber disposed inside the housing for receiving a sample container, a temperature-regulating unit and an electronic controller that controls the temperature-regulating unit, wherein the sample chamber can be heated and/or cooled by means of the temperature-regulating unit and the housing has a closable access opening for loading the sample chamber, the housing being suitable for supporting at least one further incubator stacked vertically on top of it. It is further characterized in that it has a bus system cooperating with its electronic controller and two connecting elements connected to the bus system, wherein its bus system can be connected by the first connecting element to a central control unit and by the second connecting element to a bus system of a further incubator (slave incubator), so that the electronic controller of the incubator and of the further incubator can be individually activated by the central control unit by virtue of the architecture of the bus system.

Likewise the present invention also relates to incubators that can be connected to the master incubator or another slave device. Such an incubator encompasses a housing, a sample chamber disposed inside the housing for receiving a sample container, a temperature-regulating unit and an electronic controller that controls the temperature-regulating unit, wherein the sample chamber can be heated and/or cooled by means of the temperature-regulating unit and the housing has a closable access opening for loading the sample chamber, the housing being suitable for supporting at least one further incubator stacked vertically on top of it. It is further characterized in that it has a bus system cooperating with its electronic controller and two connecting elements connected to the bus system, wherein its bus system can be connected by each of the two connecting elements to the respective bus system of a further incubator.

Obviously all advantageous configurations related to the system—even if they referred to only one incubator or to some of the incubators of the system—can be applied analogously to the two incubators described in the foregoing. In particular, the Applicant reserves the right to make individual protectable configurations of the incubators described hereinabove subject matter of a divisional application, independently of the features regarded at present as essential to the invention.

### BRIEF DESCRIPTION OF THE DRAWINGS

The inventive system as well as the inventive incubators will be explained in more detail hereinafter with reference to the drawing, wherein:

FIG. 1 shows an exemplary embodiment of an inventive system,

FIG. 2 shows a perspective view of a first exemplary embodiment of an inventive incubator with drawn drawer,

FIG. 3 shows a perspective view of individual components of the exemplary embodiment according to FIG. 2,

FIG. 4 shows a diagram of section through the exemplary embodiment according to FIGS. 2 and 3 with the drawer closed,

FIG. 5 shows an exploded diagram of the exemplary embodiment according to FIGS. 2, 3 and 4,

FIG. 6 shows a perspective view of a detail of a second exemplary embodiment of an inventive incubator, and

FIG. 7 shows an exploded diagram of a third exemplary embodiment of an inventive incubator.

### DETAILED DESCRIPTION OF THE PREFERRED EMBODIMENTS

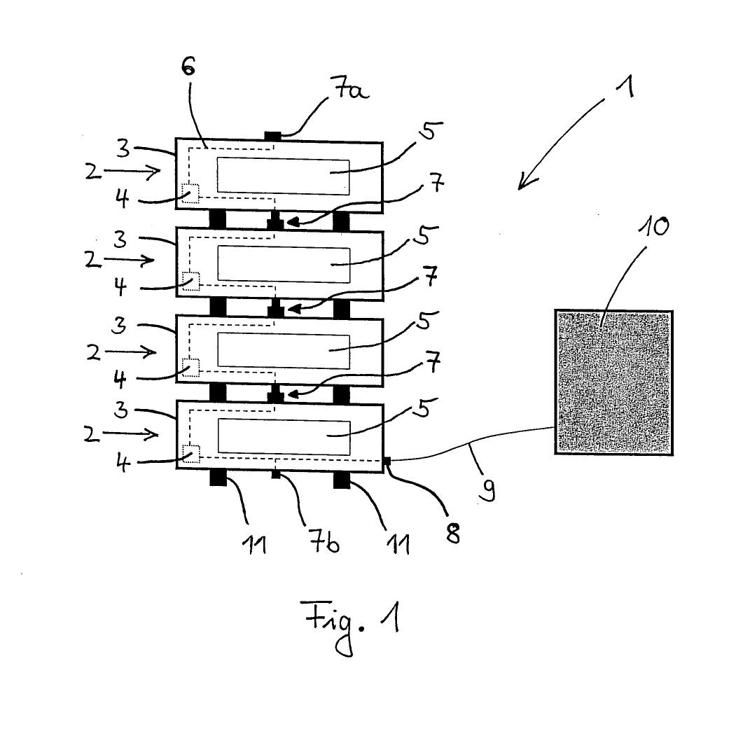

FIG. 1 shows a schematic diagram of the front view of an exemplary embodiment of an inventive system 1. The system encompasses a plurality of incubators 2, which are stacked vertically one above the other, and which respectively have a housing 3 as well as—not illustrated in FIG. 1—a sample chamber that is used to receive a sample container and that can be heated and/or cooled by means of a temperature-regulating unit. Housings 3 of the incubators each have on the front side an access opening—which can be closed by means of a shutter 5—to the sample chamber. The respective temperature-regulating unit of incubators 2 can be controlled by an electronic controller 4—indicated by dashed lines—integrated into the respective incubator 2. Each incubator 2 also has a bus system 6 (also represented by dashed lines) cooperating with its electronic controller 4. Bus systems 6 integrated into incubators 2 are connected to one another by corresponding connecting elements 7, thus forming a bus-system architecture, which is connected to a central control unit 10 via a connecting element 8 disposed on the lowermost incubator of the stack and a connecting line 9, which if necessary could also be replaced by a wirelessly functioning communication network. Alternatively, however, central control unit 10 could also be connected to connecting element 7b of the lowermost incubator. In the present figure, the lowermost incubator of the stack is used as the master incubator. Individual activation of the electronic controllers 4 of all incubators 2 is possible via central control unit 10. Connecting elements 7 have the form of plug-and-socket elements 7a, which are respectively disposed on the upper side of incubators 2 and which are connected to corresponding plug-and-socket elements 7b on the underside of incubators 2 to construct a stack. Each of the incubators 2 additionally has feet 11, which engage in corresponding cutouts (not illustrated in FIG. 1) in the upper side of the incubator disposed below. By means of these cutouts, exact positioning of the incubators—in both horizontal and vertical direction—is ensured.

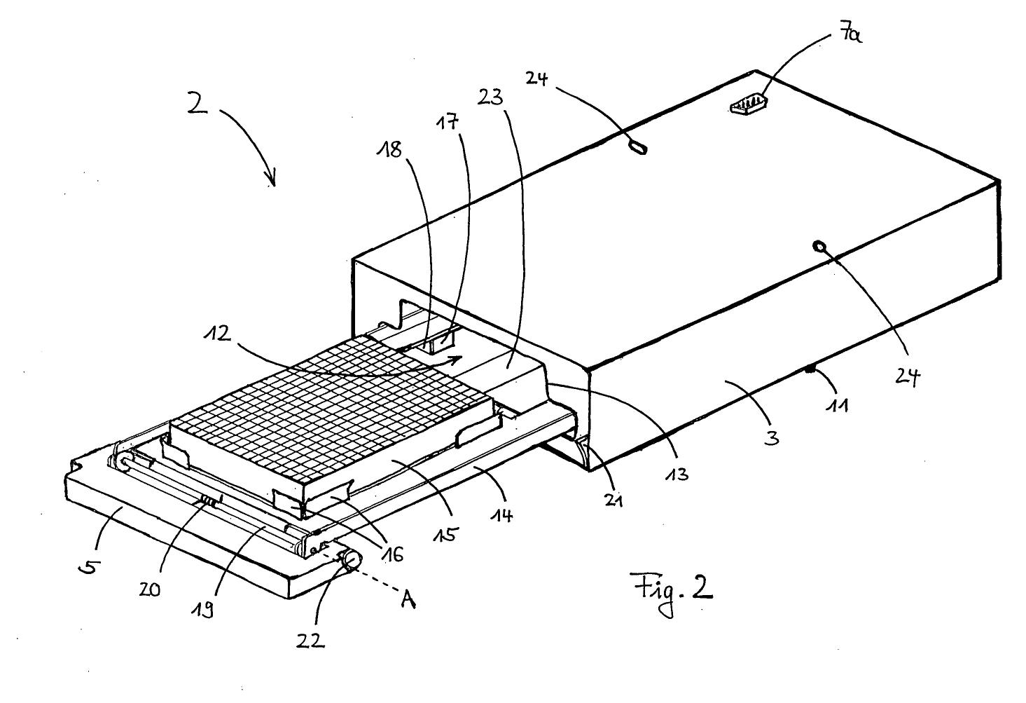

FIG. 2 represents a perspective view of a first exemplary embodiment of an inventive incubator. Incubator 2 possesses a housing 3, in which there is disposed a sample chamber 12 for receiving samples. For loading and unloading sample chamber 12, housing 3 of incubator 2 has on its front side an access opening 13, through which a drawer 14 forming the bottom of sample chamber 12 can be withdrawn and inserted. Drawer 14, which in the present figure is completely withdrawn from sample chamber 12, supports a sample container 15, which is secured against slipping by means of spring clamps 16 and strikes with a stop element 17 fastened to drawer 14 against a stop 18 fastened to housing 3 of the incubator, whereby its completely withdrawn position can be exactly defined.

Shutter 5, which is fixed on the end face of drawer 14 such that it can pivot around a horizontal spindle A, is oriented in substantially vertical position that closes access opening 13 when drawer 14 is closed, and in the present figure is pivoted out of the way into substantially horizontal position. Such pivoting—initiated by a helical spring 20, which is mounted on connecting element 19 and which is braced at one end against drawer 14 and at the other against shutter 5—takes place automatically when drawer 14 is withdrawn. Connecting element 19 forming spindle A is attached to the shutter not at the rim but—as viewed in vertical position of the shutter—at a distance from its lower and upper rims.

When drawer 14 is inserted into sample chamber 12, shutter 5 is automatically returned to a vertical position, in which it then closes access opening 13 to sample chamber 12. This is achieved by means of a curved guide 21, along which an element 22—which is preferably mounted to rotate—of shutter 5 is guided at the end of insertion of drawer 14.

Underneath sample container 15 there is disposed a heating film—not illustrated in FIG. 2—for heating the samples or sample chamber 12, which is connected to the electronic controller—also not illustrated in FIG. 2—of incubator 2 by means of a ribbon cable 23.

On the upper side of housing 3 there are disposed holders 24 for feet 11 of a further incubator. In order to prevent jamming of feet 11 of a further incubator in holders 24, these are configured in such a way that one of the holders 24 fixes foot 11 received therein in the horizontal plane, while the other holder 24 supports the foot received therein loosely in the direction connecting the two holders. This is evident in FIG. 2 by the oval configuration of holder 24 illustrated on the left. Also disposed on the upper side of incubator 2 is a plug-and-socket element 7a, via which the bus system cooperating with the electronic controller of incubator 2 can be connected to the bus system of a further incubator, when this is stacked on the incubator.

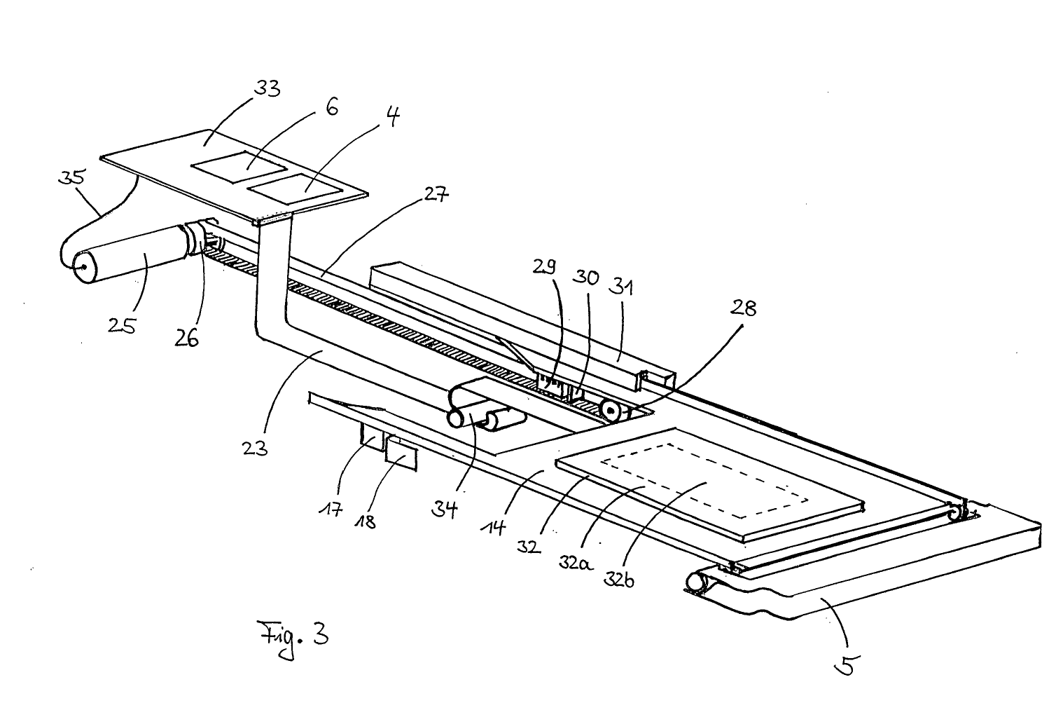

FIG. 3 shows a perspective view of an exemplary embodiment of a drawer system of an inventive incubator. Numerous elements of the incubator, such as its housing in particular, are not illustrated therein, for reasons of clarity. Drawer 14 is driven by an electric motor 25 and a toothed-belt drive 26, 27, 28. Toothed belt 27 thereof is guided over two deflecting rollers 26, 28, the first of which is driven by electric motor 25, which is mounted in fixed position on the housing. Toothed belt 27 is clamped on drawer 14 by means of a clamping block 29 fastened thereon, and clamping block 29 additionally serves—besides first stop element 17—as a second stop element of drawer 14.

When drawer 14 is completely withdrawn, the two stop elements 17, 29 bear respectively against incubator stops 18, 30 fastened to the housing. Drawer 14 is guided on both sides in linear guides 31, one of which is not illustrated in FIG. 3, for reasons of clarity.

A heating film 32 for heating the sample chamber is disposed on the drawer and connected via a ribbon cable 23 to a circuit board 33. Heating film 32 is designed as a two-zone heating film, a first zone 32a of which heats the outer region while a second zone 32b heats the inner region of heating film 32. Starting from heating film 32, ribbon cable 23 is first passed from above through a cutout—not illustrated—in drawer 14, which otherwise has a substantially continuous surface, is deflected two times in the region of a clamping roller 34 and is fastened by clamping roller 34 to the bottom of the housing in such a way that it does not interfere with the movement of drawer 14.

Electronic controller 4—schematically illustrated—for activating heating film 32 is disposed on circuit board 33, as is bus system 6 of the incubator. Via connecting cable 35, electric motor 25 can also be controlled by electronic controller 4. This controller is also connected to bus system 6, which has all of the functional features described hereinabove and is connected to two connecting elements (not illustrated in FIG. 3) of incubator 2. Via bus system 6, electronic controller 4 can be activated by a central control unit, which is connected directly or indirectly via one of the connecting elements.

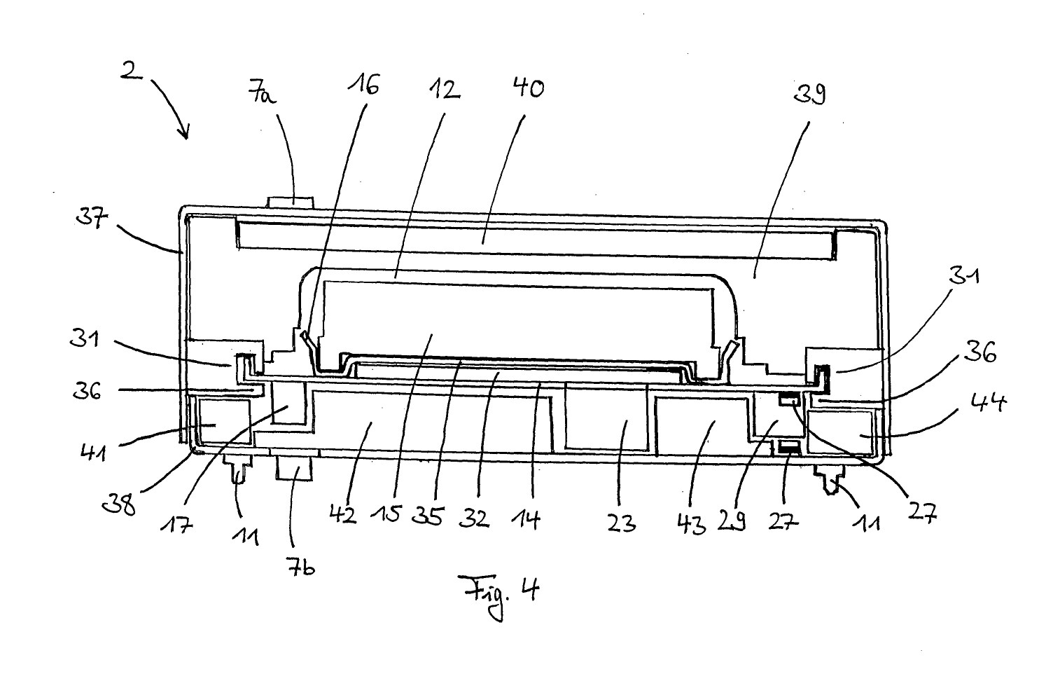

FIG. 4 shows a sectional drawing of an exemplary embodiment of an inventive incubator 2 in a cross-sectional plane through sample chamber 12. Inside sample chamber 12 there is disposed a sample container 15, which contains one or more samples—not illustrated. Sample container 15 is received in a holder 35, which is fastened to drawer 14 and which on its side rims forms spring clamps 16, which fix sample container 15 laterally in holder 35. Between drawer 14 and holder 35 there is disposed a heating film 32, which is provided for heating sample chamber 12 and is connected via a ribbon cable 23 to the electronic controller of incubator 2. Good heat transfer from the heating film to the sample container—and thus also to the sample contained therein—is ensured by substantially full-surface contact of heating film 32 with holder 35, which in turn bears on the underside of sample container 15. Drawer 14 is guided on both sides by linear guides 31, which are fastened to the housing and both of which fix drawer 14 in vertical direction. In horizontal direction, and transverse to the direction of movement of drawer 14, drawer 14 is fixed in linear guide 31 illustrated on the right and is supported loosely in linear guide 31 illustrated on the left, in order to allow for some thermal expansion of drawer 14 in this direction. In its rim region, drawer 14 rests on a support 36, which simultaneously forms part of linear guide 31, and can be moved horizontally thereon. In the interests of the best possible sliding ability of drawer 14, a particularly favorable combination has proved to be stainless steel for drawer 14 and plastic, preferably PETP-TX containing Teflon, for linear guide 31 or support 36. Also evident in FIG. 4 are the two stop elements 17, 29 fixed on drawer 14, stop element 29 shown on the right in the drawing simultaneously serving as the clamping block for connecting drawer 14 to toothed belt 27 of the drive unit of the drawer. The housing encompasses an upper housing part 37 and a lower housing part 38, and on its upper side and underside has plug-and-socket elements 7a, 7b respectively for connecting its bus system to the bus system of an incubator disposed above or below it or to a central control unit. For insulation purposes, sample chamber 12 is provided at the top with a sandwich structure comprising a first insulating layer 39 of solid plastic and a second vacuum-insulated layer 40. Insulating layer 39 of solid plastic also insulates sample chamber 12 at the side, somewhat limited thermal insulation being tolerated in the region of linear guide 31 with support 36 for drawer 14. Further elements 41, 42, 43, 44 of solid plastic insulate the sample chamber at the bottom. Here also a second vacuum-insulated layer can be provided if necessary underneath elements 41 to 44.

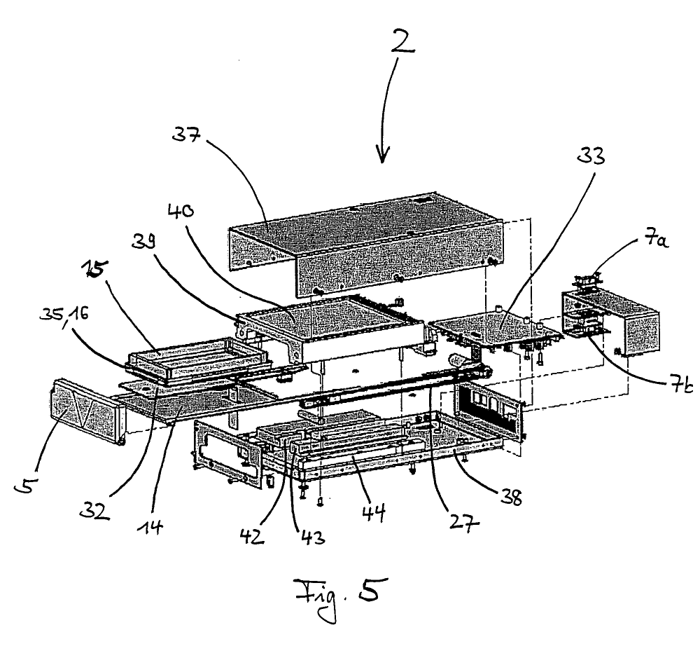

For better clarity, FIG. 5 shows an exploded diagram of the first exemplary embodiment of the inventive incubators.

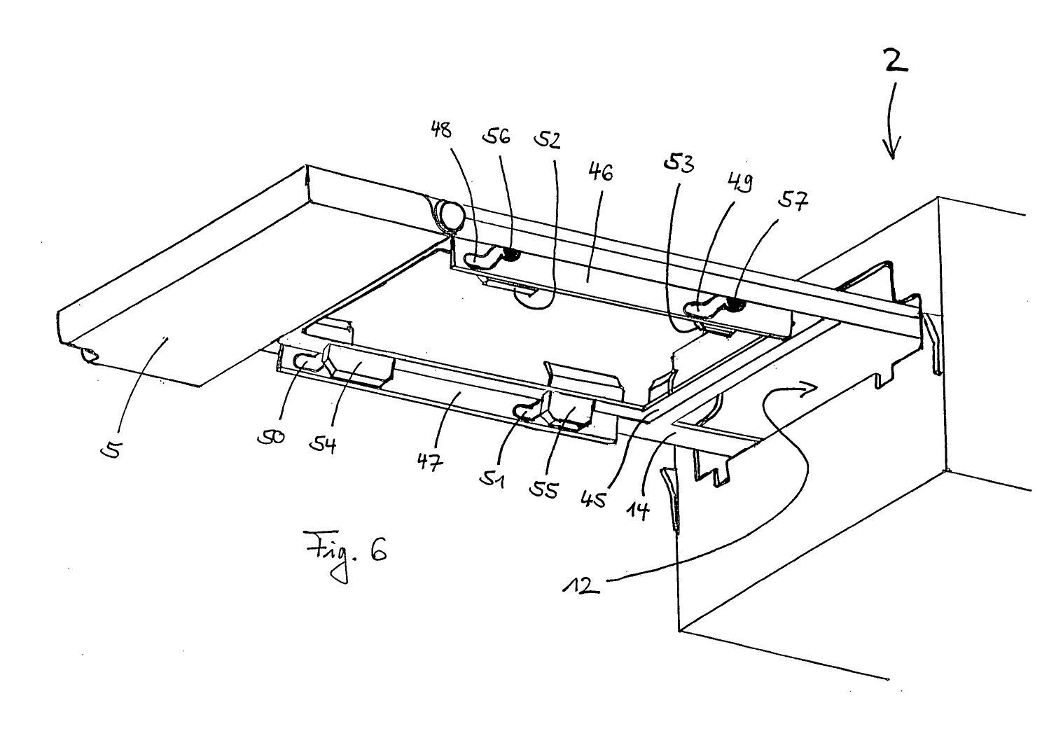

FIG. 6 shows a perspective diagram, viewed obliquely from the bottom right, of a second exemplary embodiment of an inventive incubator 2, wherein drawer 14 does not have a continuous surface but is constructed in the form of a frame. Along its inner periphery this frame supports a holder 45 for a sample container. Also fastened to drawer 14 is a slide guide, which on the two sides respectively encompasses a base plate 46 or 47 with guides 48, 49 or 50, 51 formed therein. Holder 45 has four guide-pin retainers 52, 53, 54, 55, on each of which there is formed a guide pin 56, 57 pointing in the direction of the slide guide. For perspective reasons, the two guide pins 56, 57 that engage in guides 48, 49 of the slide guide are visible. Inside incubator 2 there is also provided—but not illustrated—a stop for holder 45 or for the sample container disposed and fixed in holder 45. This stop is disposed in such a way that holder 45 or the sample container already strikes it before the operation of closing of drawer 14 has ended. Thereby holder 45 and the sample container disposed therein are displaced horizontally relative to the drawer during the last portion of the operation of closing of drawer 14, and the slide guide ensures that holder 45 moving horizontally relative to drawer 14 is lowered vertically by guide pins 56, 57 moving in guides 48 to 51. In this way the sample container can be lowered vertically to the bottom of sample chamber 12 or to an intermediate plate disposed therein during the operation of closing of drawer 14.

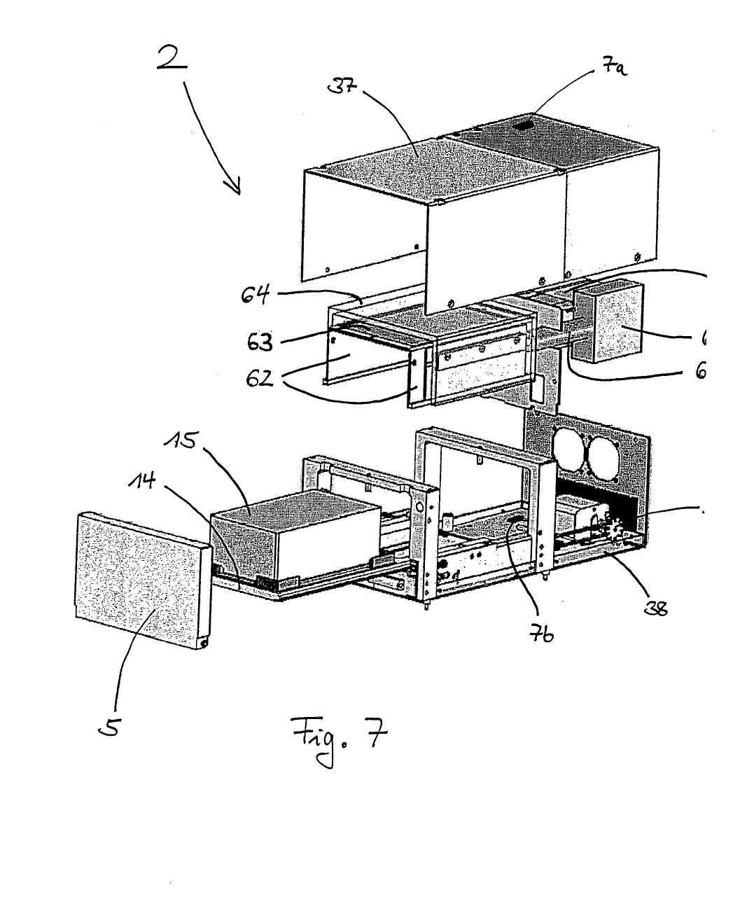

Finally, FIG. 7 also shows an exploded diagram of a further exemplary embodiment of an inventive incubator 2, whose housing encompasses an upper housing part 37 and a lower housing part 38. The sample disposed in a sample container 15 can be inserted into and again withdrawn from the housing of incubator 2 by means of a drawer 14 driven by a toothed-belt drive 58, the front access opening necessary for this purpose being closable by a shutter 5 disposed on the front side of the housing. The sample chamber can be both cooled and heated in a temperature range of between +4° C. and +80° C. (given room temperature in the laboratory) by a combined cooling/heating group 59. The combined cooling/heating group is in thermally conductive communication with side walls 62 of the sample chamber via a cooling/heating flange 60 and heat pipes 61 respectively. Cover 63 of the sample chamber is not directly cooled. The sample chamber is insulated at the top and sides with a sandwich structure 64, which—illustrated in transparent form without details—is provided from inside to outside with solid plastic first, followed by closed-cell foam rubber and finally vacuum insulation. The electronic controller—not illustrated—of the incubator is connected via a bus system to plug-and-socket elements 7a, 7b, which are used for connecting the bus system of incubator 2 to bus systems of further incubators.

We claim:

1. A system (1) comprising:
a plurality of incubators (2), each encompassing a housing (3), a sample chamber (12) disposed inside the housing (3) for receiving a sample container (15), a temperature-regulating unit and an electronic controller (4) that controls the temperature-regulating unit,
wherein the sample chambers (12) of the incubators (2) can be heated and/or cooled by the temperature-regulating units,
the housings (3) of the incubators (2) each having a closable access opening (13) for loading and unloading the sample chamber (12),
the housings (3) of the incubators (2) permitting vertical stacking of the incubators (2),
the incubators (2) respectively having a bus system (6) cooperating with their electronic controllers (4), wherein the bus systems (6) of the incubators (2) are connected to one another via connecting elements (7), and
the system (1) further comprising a central control unit (10) connected to the bus system (6) of one of the incubators (2) of the system (1) to define a master incubator, and the electronic controllers (4) of all incubators (2) of the system (1) can be activated individually by the central control unit (10) by virtue of the architecture of the bus system.

2. A system according to claim 1, wherein two neighboring incubators (2) can be connected to one another by two respective connecting elements in the form of mating plug-and-socket elements (7a, 7b), wherein the plug-and-socket elements (7a, 7b) of at least some of the incubators (2) are disposed opposite one another on an upper side and underside of the housing (3), in such a way that they form a plug-and-socket connection with likewise mating plug-and-socket elements (7a, 7b) of the incubator (2) disposed below them and/or stacked on top of them.

3. A system according to claim 1, wherein at least some of the incubators (2) have a drawer (14) for loading and unloading the sample chamber (12), which drawer is guided in linear guides (31) and by a motor (25) can be withdrawn through the access opening (13) from the housing (3) of the incubator (2) and reinserted therein.

4. A system according to claim 3, wherein the drawer (14) in its withdrawn end position is stopped against at least one stop (18, 30) of the incubator (2).

5. A system according to claim 4, wherein the access opening (13) in the housing (3) can be closed by a shutter (5) fastened on the end face of the drawer (14).

6. A system according to claim 5, wherein the shutter (5) occupies a substantially vertical position that closes the access opening (13) when the drawer (14) is inserted and, at a distance from its lower or upper rim as viewed in vertical position, is fastened pivotally to the drawer (14) around a horizontal spindle (A), such that it pivots out of the way into substantially horizontal position when the drawer (14) is withdrawn.

7. A system according to claim 1, wherein the temperature-regulating unit of at least one incubator of the system further comprises a heating film disposed on the bottom of the sample chamber (12).

8. A system according to claim 7, wherein the heating film (32) is a two-zone heating film (32) or the temperature-regulating unit comprises at least two heating films, with which at least two zones (32a, 32b) respectively can be heated separately.

9. A system according to claim 1, wherein the temperature-regulating unit of at least one of the incubators (2) comprises a cooling group (59) for cooling the sample chamber (12).

10. A system according to claim 1, wherein the temperature-regulating unit of at least one of the incubators (2) comprises a combined cooling/heating group (59) for cooling and heating the sample chamber (12).

11. A system according to claim 10, wherein only the bottom and/or side walls (62) of the sample chamber (12) can be directly cooled.

12. A system according to claim 11, wherein the bottom and/or side walls (62) of the sample chamber is/are spatially separated from the cooling group or the combined heating/cooling group (59) and are in communication therewith by heat pipes (61).

13. A system according to claim 1, wherein the sample chamber (12) of at least one of the incubators (2) further comprises insulation in the form of a sandwich structure comprising, from inside to outside, at least two different insulation types (38, 40), the outer of which is designed as vacuum insulation (40).

14. A system according to claim 13, wherein the sample chamber of the at least one incubator is insulated from inside to outside by a sandwich structure (64) comprising solid plastic, closed-cell foam rubber and vacuum insulation.

15. A system according to claim 3, wherein the drawer (14) of at least one of the incubators (2) encompasses a holder (45) for a sample container (15), the holder being guided by a slide guide (46-51, 56, 57), and the slide guide (46-51, 56, 57) bringing about vertical lowering of the holder (45) during the last portion of the horizontal insertion travel of the drawer into the sample chamber (12) in order to deposit the sample container (15) on an intermediate plate or on the bottom of the sample chamber (12).

16. A system according to claim 15, wherein an underside of the intermediate plate is geometrically adapted to a bottom of the sample chamber (12) being heated and/or cooled by the temperature-regulating unit and the geometry of its upper side is adapted to a sample container (15) to be introduced into the sample chamber (12).

17. A system according to claim 15, wherein the intermediate plate is disposed in the sample chamber (12) such that it can move in the horizontal plane and can be excited to vibrations in the horizontal plane by a drive system.

18. An incubator in the form of a master incubator comprising:
a housing (3),
a sample chamber (12) disposed inside the housing (3) for receiving a sample container (15),
a temperature-regulating unit and an electronic controller (4) that controls the temperature-regulating unit,
wherein the sample chamber (12) can be heated and/or cooled by the temperature-regulating unit and the housing (3) has a closable access opening (13) for loading and unloading the sample chamber (12),
the housing (3) being suitable for supporting at least one further incubator (2) stacked vertically on top of it,
a bus system (6) cooperating with the electronic controller (4) and two connecting elements (7a, 7b, 8) connected to the bus system (6), the bus system (6) can be connected by a first connecting element (7b, 8) to a central control unit and by a second connecting element (7a) to a bus system (6) of a further incubator in the form of a slave incubator (2), so that the electronic controller (4) of the incubator (2) and of the further incubator (2) can be individually activated by the central control unit (10) by virtue of the architecture of the bus system.

19. An incubator in the form of a slave incubator comprising:
a housing (3),
a sample chamber (12) disposed inside the housing (3) for receiving a sample container (15),
a temperature-regulating unit and an electronic controller (4) that controls the temperature-regulating unit,
wherein the sample chamber (12) can be heated and/or cooled by means of the temperature-regulating unit and the housing (3) at its front side has a closable access opening (13) for loading and unloading the sample chamber (12),
the housing (3) being suitable for supporting at least one further incubator (2) stacked vertically on top of it,
the incubator (2) further comprising a bus system (6) cooperating with its electronic controller (4) and two connecting elements (7a, 7b) connected to the bus system (6), wherein the bus system (6) of the incubator (2) can be connected by each of the two connecting elements (7a, 7b) to the respective bus system (6) of a further incubator (2).
